(19) United States
(12) Patent Application Publication
(10) Pub. No.: US 2024/0350690 A1
(43) Pub. Date: Oct. 24, 2024

(54) ELECTROLESS ANTIPATHOGENIC COATING

(71) Applicant: Coventya, Inc., Brooklyn Heights, OH (US)

(72) Inventors: Ambrose Schaffer, Canajoharie, NY (US); Jean Laplante, Oriskany, NY (US); Matthew Wojcik, Utica, NY (US)

(21) Appl. No.: 18/685,635

(22) PCT Filed: Oct. 25, 2022
(86) PCT No.: PCT/US2022/047706

§ 371 Date: Feb. 22, 2024

Related U.S. Application Data
(60) Provisional application No. 63/284,274, filed on Nov. 30, 2021.

(51) Int. Cl. A61L 2/238; A01N 59/20; A01P 1/00; A61L 101/26; C22C 19/00; C22C 19/03; C23C 18/16; C23C 18/50

(52) U.S. Cl. CPC A61L 2/238; A01N 59/20; A01P 1/00; C22C 19/002; C22C 19/03; C23C 18/1646; C23C 18/50; A61L 2101/26

(57) ABSTRACT

An electroless nickel-copper-phosphorous coating includes at least about 30% by weight Cu: about 5% to about 15% by weight P; and the balance Ni, with incidental impurities. The electroless nickel-copper-phosphorus coating exhibits antipathogenic properties and enhanced wear when deposited on a substrate.

### TECHNICAL FIELD

This application relates to electroless nickel-copper-phosphorous plating baths, methods for forming electroless antipathogenic coatings on work pieces, and electroless antipathogenic coatings so formed.

### BACKGROUND OF THE INVENTION

SARS-CoV-2 is the pathogen responsible for coronavirus disease 2019 (COVID-19). The prevalence of COVID-19 has led to an increased awareness of the spread of disease based on the ability of pathogens to survive on surfaces and infect a person that comes into contact with the surface while the pathogen remains viable. There is a whole industry predicated on manufacturing disinfectants to spray or wipe on surfaces to eliminate pathogen viability, making the surfaces safe for contact. There are also substances that can be applied to surfaces that maintain the disinfecting properties for some length of time, but the materials must be reapplied to maintain efficacy.

Rather than apply a disinfectant to a surface, it is also possible to modify the surface such that the surface itself is the disinfecting medium. This can be accomplished, for example, by coating the surface with a layer of metal that has antipathogenic properties. The use of a metal antipathogenic coating is desirable because a coating of metal can provide the antipathogenic properties without forging a heavy piece (e.g., plating on plastic) or one can use a less costly metal for primary fabrication (iron) and then have the contact surface coated with a layer of the metal that has antipathogenic properties.

The ability of metals to exhibit biocidal effects is known as the oligodynamic effect. The antipathogenic effectiveness varies from metal to metal, but the effectiveness is unequivocal, in some cases even at low concentrations, and the two metals with the greatest effectiveness are copper and silver. While they both have good antipathogenic tendencies, they tend to have poor wear characteristics because they are soft metals when compared to nickel, chromium or iron, which have inferior antipathogenic properties.

It is desirable to have the entirety of a substrate or work piece encapsulated with a metallic antipathogenic coating so that the entire surface of the substrate can provide disinfecting properties anywhere the substrate is contacted. One method for achieving such encapsulation is by plating the substrate or work piece. However, not all kinds of plating yield positive results. Electroplating has self-shielding, current density distribution and line of sight issues that could potentially yield areas of non-coverage. Immersion deposition methodologies, often referred to as substitution or displacement plating, could provide for total coverage, but deposits are very thin, often with adhesion issues. Moreover, deposition can only occur when the depositing a metal is more noble than the substrate. Electroplating requires a conductive substrate and immersion deposition requires a metallic substrate.

Autocatalytic electroless plating does not suffer any of these issues. Autocatalytic electroless plating allows for coverage of a variety of substrates, both metallic (iron and copper bearing) as well as plastic (plating on plastic) and the electroless deposit can be robust enough to allow for considerable wear from repeated contact. One metal that offers a robust combination of wear and application is electroless nickel, which is deposited typically as a nickel phosphorous alloy. While nickel does have some antipathogenic properties, it is not as effective as other metals.

### SUMMARY OF THE INVENTION

Embodiments described herein relate to an electroless nickel-copper-phosphorous plating bath that deposits an alloy of nickel-copper-phosphorous (NiCuP) having antipathogenic properties and enhanced wear. Advantageously, the electroless nickel-copper-phosphorous bath can provide a uniform electroless nickel-copper-phosphorous deposit that can completely encapsulate both metallic and non-metallic substrates.

In some embodiments, the aqueous electroless nickel-copper-phosphorous plating bath can include about 2.0 g/l to about 8.0 g/l Ni: about 150 ppm to about 1500 ppm Cu; and about 10 g/l to about 50 g/l hypophosphorous reducing agent. The electroless nickel-copper-phosphorous bath can provide a uniform electroless nickel-copper-phosphorous alloy deposit on the substrate that is antipathogenic and/or oligodynamic.

In some embodiments, the concentration of Ni in the bath relative to the concentration of Cu is such that the electroless nickel-copper-phosphorous alloy deposit includes about 30% to about 85% Cu.

In some embodiments, the Ni concentration in the bath is about 3 g/L to about 6 g/L. The Ni can be provided in the bath by dissolving a water soluble nickel salt in the bath. The water soluble nickel salt can be selected from nickel sulfate, nickel chloride, nickel methane sulfonate, nickel sulfamate, nickel fluoroborate, or combinations thereof.

In some embodiments, the Cu concentration in the bath is about 400 ppm to about 1000 ppm. The Cu can be provided in the bath by dissolving a water soluble copper salt in an aqueous solution. The copper salt can be selected from copper sulfate, copper chloride, copper methane sulfonate, copper sulfamate, copper fluoroborate, or combinations thereof.

In some embodiments, the bath can have a neutral to alkaline pH. For example, the bath can have a pH of about 8 to about 9.

In some embodiments, the bath can include about 10 g/L to about 50 g/L of at least one organic acid complexing agent. In some embodiments, the organic acid complexing agent may be a carboxylic acid such as at least one of malic acid, lactic acid, or succinic acid.

In other embodiments, the bath can include at least one of a chelating agent, stabilizer, or pH buffer.

In some embodiments, the baths can consist essentially of water, about 3 g/L to about 6 g/L of Ni, about 10 g/L to about 50 g/L hypophosphorous reducing agent, about 400 ppm to about 1000 ppm Cu, less than about 25 g/L of a combination of organic acid complexing agents selected from the group consisting of malic acid, lactic acid, and succinic acid, an optional stabilizer, and an optional pH adjuster. The bath can have a pH of about 8 to about 9.

Other embodiments described herein, relate to a method of forming an electroless antipathogenic coating on a surface of a substrate. The method can include providing the substrate and contacting the surface of substrate with the aqueous electroless nickel-copper-phosphorous plating bath described herein.

In some embodiments, the surface of the substrate is contacted with the aqueous electroless nickel-copper-phosphorous plating bath by submerging at least a portion of the substrate in the bath. The bath can have a temperature during electroless plating of the surface of the substrate of about 160″F to about 195° F.

In some embodiments, the surface of the substrate can be in contact with the bath for at least 1 hr. The coating formed using the bath can have a thickness of at least about 50 microinches.

Still other embodiments described herein relate to an electroless nickel-copper-phosphorous alloy coating that includes at least about 30% by weight Cu; about 5% to about 15% by weight P; and the balance Ni, with incidental impurities.

In some embodiments, the coating an include at least about 40%, at least about 50%, or at least about 60% by weight Cu and have a thickness of at least about 50 microinches. The coating can be formed using the bath and/or the method described herein.

In some embodiments, the coating can be biocidal, antimicrobial, and/or destroy or inhibit the growth microorganisms, such as bacteria, fungi, and viruses.

In other embodiments, the coating can be antipathogenic and destroy or inhibit the growth of bacteria, such as Staphylococcus aureus or methicillin-resistant Staphylococcus aureus.

In still other embodiments, the coating can inhibit bio-film formation.

### DETAILED DESCRIPTION

In the specification and the claims, which follow, reference will be made to a number of terms, which shall be defined to have the following meanings.

The singular forms “a”, “an” and “the” include plural referents unless the context clearly dictates otherwise.

“Optional” or “optionally” means that the subsequently described event or circumstance may or may not occur, and that the description includes instances where the event occurs and instances where it does not.

Approximating language, as used herein throughout the specification and claims, may be applied to modify any quantitative representation that could permissibly vary without resulting in a change in the basic function to which it is related. In some instances, the approximating language may correspond to the precision of an instrument for measuring the value.

As used herein, the term “about” refers to a measurable value such as a parameter, an amount, a temporal duration, and the like and is meant to include variations of +/−15% or less, preferably variations of +/−10% or less, more preferably variations of +/−5% or less, even more preferably variations of +/−1% or less, and still more preferably variations of +/−0.1% or less of and from the particularly recited value, in so far as such variations are appropriate to perform in the invention described herein. Furthermore, it is also to be understood that the value to which the modifier “about” refers is itself specifically disclosed herein.

As used herein, the terms “comprises” and/or “comprising,” specify the presence of stated features, integers, steps, operations, elements, and/or components, but do not preclude the presence or addition of one or more other features, integers, steps, operations, elements, components, and/or groups thereof.

As used herein, the term “substantially free” or “essentially free” if not otherwise defined herein for a particular element or compound means that a given element or compound is not detectable by ordinary analytical means that are well known to those skilled in the art of metal plating for bath analysis. Such methods typically include atomic absorption spectrometry, titration, UV-Vis analysis, secondary ion mass spectrometry, and other commonly available analytically techniques.

The terms “plating” and “deposit” or “deposition” are used interchangeably throughout this specification.

The terms “composition” and “bath” and “electrolyte” and “solution” are used interchangeably throughout this specification.

Embodiments described herein relate to an electroless nickel-copper-phosphorous plating bath that deposits an alloy of nickel-copper-phosphorous (NiCuP) having antipathogenic or oligodynamic properties, such as antimicrobial, anti-fungal, and anti-biofilm properties, as well as enhanced wear. Advantageously, the electroless nickel-copper-phosphorous bath can provide a uniform electroless nickel-copper-phosphorous deposit that can completely encapsulate both metallic and non-metallic substrates.

In some embodiments, the electroless nickel-copper-phosphorous plating bath used to form the electroless nickel-copper-phosphorous coating includes Ni ions, Cu ions, a hypophosphorous reducing agent, and optionally at least of one of a complexing agent, chelating agent, stabilizer, and/or pH buffer. For example, the aqueous electroless nickel-copper-phosphorous plating bath can include about 2.0 g/L to about 8.0 g/L Ni; about 150 ppm to about 1500 ppm Cu; and about 10 g/L to about 50 g/L hypophosphorous reducing agent. The electroless nickel-copper-phosphorous bath provides a uniform electroless nickel-copper-phosphorous alloy deposit on the substrate that is antimicrobial, antipathogenic and/or oligodynamic.

In some embodiments, the concentration of Ni in the bath relative to the concentration of Cu is such that the electroless nickel-copper-phosphorous alloy deposit includes about 20% to about 90% Cu or about 30% to about 85% Cu or about 50% to 70% Cu.

In some embodiments, the Ni concentration in the bath can be about 2 g/L to about 8 g/L or about 3 g/L to about 6 g/L. The Ni can be provided in the bath by dissolving a water soluble nickel salt in the bath. The water-soluble nickel salt can include those which are soluble in the plating bath and which can yield an aqueous solution of a predetermined concentration. In one embodiment, the nickel salt may be selected from the group consisting of nickel sulfate, nickel chloride, nickel bromide, nickel iodide, nickel acetate, nickel malate, a nickel hypophosphite, nickel methane sulfonate, nickel sulfamate, nickel fluoroborate, and combinations thereof.

In some embodiments, the Cu concentration in the bath is 150 ppm to about 1500 ppm or about 400 ppm to about 1000 ppm. The Cu can be provided in the bath by dissolving a water soluble copper salt in an aqueous solution. The copper salt may be selected from the group consisting of copper sulfate, copper chloride, copper methane sulfonate, copper sulfamate, copper fluoroborate, and combinations thereof.

The hypophosphorous reducing agent used in the bath can include any of a variety of hypophosphorous reducing agents used in known types of the electroless nickel plating baths. In some embodiments, the hypophosphorous reducing agent is selected from the group consisting of sodium hypophosphite, potassium hypophosphite, ammonium hypophosphite, and combinations thereof.

The concentration of the hypophosphorous reducing agent in the electroless nickel plating can vary depending on the type of hypophosphorous reducing agent and can be adjusted to vary the concentration of the phosphorous in the electroless nickel-copper-phosphorous coating that is formed using the bath. In some embodiments, the concentration of the hypophosphorous reducing agent in the electroless nickel-phosphorous plating bath can be about 10 g/L to about 50 g/L, or about 15 g/L to about 35 g/L, or about 20 g/L to about 30 g/L. In other embodiments, the concentration of the hypophosphorous reducing agent in the electroless nickel-copper-phosphorous plating bath can be about 25 g/L.

In some embodiments, a complexing agent or a mixture of complexing agents may be included in the electroless nickel-copper-phosphorous plating bath. Complexing agents as used herein can also include chelating agents. The complexing agents and/or chelating agents generally retard the precipitation of nickel ions or copper ions from the plating solution as insoluble salts, such as phosphites, by forming a more stable nickel complex with the nickel ions or more stable copper complex with the copper ions and provide for a moderate rate of the reaction of nickel or copper precipitation.

The complexing agents and/or chelating agents can be included in the plating bath in amounts sufficient to complex the nickel ions or copper ions present in the bath and to further solubilize the hypophosphite degradation products formed during the plating process. In some embodiments, the complexing agents and/or chelating agents are provided in the electroless nickel-phosphorous plating bath at amounts less than about 30 g/L or less than about 25 g/L, or from about 20 g/L to about 30 g/L.

A variety of complexing agents, used in known electroless nickel plating solutions, may be used. Specific examples of the complexing agents may include monocarboxylic acids, such as glycolic acid, lactic acid, gluconic acid or propionic acid, dicarboxylic acids, such as malic acid, malonic acid, succinic acid, tartaric acid, oxalic acid or adipic acid, aminocarboxylic acids, such as glycine or alanine, ethylene diamine derivatives, such as ethylenediamine tetraacetate, versenol (N-hydroxyethyl ethylenediamine-N,N′,N′-triacetic acid) or quadrol (N,N,N′, N′-tetrahydroxyethyl ethylene diamine), phosphnic acids, such as 1-hydroxyethane-1,1-diphosphonic acid, ethylene diamine tetramethylene phosphonic acid and water-soluble salts thereof. The complexing agents may be used either alone or in combination.

Some complexing agents, such as acetic acid or succinic, for example, may also act as a pH buffering agent, and the appropriate concentration of such additive components can be optimized for any plating bath after consideration of their dual functionality.

In some embodiments, at least one pH buffer, complexing agent, or chelating agent can be selected from the group consisting of an acetic acid, formic acid, succinic acid, malonic acid, an ammonium salt, lactic acid, malic acid, citric acid, glycine, alanine, glycolic acid, lysine, aspartic acid, ethylene diamine tetraacetic acid (EDTA), and combinations thereof. In some embodiments, mixtures of 2 or more of the above pH buffers, complexing agents, and/or chelating agents can be used in the electroless nickel-copper-phosphorous plating bath described herein.

In some embodiments, the electroless nickel-copper-phosphorous plating bath can include about 20 g/L to about 30 g/L of a combination of organic acid complexing agents selected from the group consisting of malic acid, lactic acid, and succinic acid. In other embodiments, the electroless nickel-copper-phosphorous plating bath can include less than about 25 g/L of a combination of organic acid complexing agents selected from the group consisting of malic acid, lactic acid, and succinic acid.

The plating bath may also contain, in addition to the above components, additives with various kinds of purposes so long as the properties of the plating bath are not deteriorated. For example, the bath can include a stabilizer, such as lead acetate, at a concentration of about 0.0004 g/L to about 0.0007 g/L. Other stabilizers can also be used besides or in addition to lead acetate including those metal cations that act as catalytic poisons, such as Pb, Bi, Sn, In, salts or compounds thereof and combinations thereof.

In other embodiments, the electroless nickel-copper-phosphorous plating bath can be free of or substantially free of additives, such as surfactants, which can have a negative effect on the antipathogenic properties, oligodynamic properties, wear, and/or surface morphology of the electroless nickel-copper-phosphorous deposit. Surfactants including organic compounds from the class of fatty acids and water soluble salts thereof, amino compounds, and sulfates and sulfonates of fatty acids and fatty alcohols are typically employed in electroless nickel plating baths to modify the inherent properties of the plating bath itself or the quality of the nickel deposit. These additives, however, can potentially form colloids with other components of the bath, such as free Ni or Cu ions, that can produce plating defects. Advantageously, the plating bath described herein can be free of or substantially free of such additives to minimize defects, such as micro-pitting and particle deposition, in the electroless nickel-copper-phosphorous deposit.

The aqueous electroless nickel-copper-phosphorous plating baths can be operated or maintained at a neutral to alkaline pH. For example, the bath can have a pH of about 7 to about 10, more preferably about 8 to about 9 during electroless nickel-copper-phosphorous plating of a substrate.

At least one pH adjustment agent can be used to adjust the pH to the above range. When the pH of the bath is too high, it can be adjusted by adding, for example, an acid. When the pH of the bath is too low, it can be adjusted by adding, for example, ammonium hydroxide.

The stability of the operating pH of the plating bath can be controlled by the addition of various buffer compounds such as acetic acid, propionic acid, boric acid, or the like, in amounts up to about 30 g/L with amounts of from about 2 g/L to about 30 g/L being typical. As noted above, some of the buffering compounds, such as acetic acid and succinic acid may also function as complexing agents.

In other embodiments, the bath can consist essentially of water, about 3 g/L to about 6 g/L of Ni, about 10 g/L to about 50 g/L hypophosphorous reducing agent, about 400 ppm to about 1000 ppm Cu, less than about 25 g/L of a combination of organic acid complexing agents selected from the group consisting of malic acid, lactic acid, and succinic acid, an optional stabilizer, and an optional pH adjuster.

In accordance with the methods described herein, a substrate or work piece can be plated with the electroless nickel-copper-phosphorous plating bath to provide an electroless nickel-copper-phosphorous alloy deposit or coating on the substrate that has antipathogenic properties or oligodynamic properties and enhanced wear.

The substrate can include any substance or material. In some embodiments, the substrate may be a substance on which electroless nickel-copper-phosphorous alloy can be deposited. The substrate can also include a substance or material that has at least one surface upon which an electroless nickel-copper-phosphorous alloy can be deposited. The substrate may include, for example, metal, plastic, paper, glass, ceramic, textile, rubber, polymer, composite material, or any combination of substrates. The substrate can also include a portion of at least one of an electronic, food or agricultural product, medical device, etc.

By way of example, the substrate can be a medical device, such as a catheter, an endotracheal tube, a tracheostomy tube, a wound drainage device, a wound dressing, a stent, an implant, an intravenous catheter, a suture, a shunt, a gastrostomy tube, medical tubing, cardiovascular products, heart valves, pacemaker leads, a guidewire, or urine collection device.

The substrate can be plated using the electroless nickel-copper-phosphorous plating bath by contacting the substrate with or immersing the substrate in the plating bath for a duration time effective to form an electroless nickel-copper-phosphorous coating or deposit at a desired thickness on at least a portion of the surface of the substrate.

In some embodiments, the substrate can be cleaned or pre-processed prior to plating using a suitable precleaner. During plating, the bath can be maintained at a bath temperature about 150° F. to about 200° F., e.g., or about 160° F. to about 195° F. The duration of contact of the electroless nickel-copper-phosphorous plating bath with the substrate being plated will determine the thickness of the electroless nickel-copper-phosphorous coating. Typically, a contact time can range from as little as about one minute to several hours or even several days, for example, about 60 to about 120 minutes. The coating formed using the bath can have a thickness of at least about 50 microinches or at least about 100 microinches up to about 500 microinches or 1,000 microinches or 2,000 microinches.

During the deposition of the electroless nickel-copper-phosphorous deposit or coating, mild to severe agitation can be employed. The mild agitation can be, for example, a mild air agitation, mechanical agitation, bath circulation by pumping, rotation of a barrel for barrel plating, rotation of mandrel for disk plating, etc. The electroless nickel-copper-phosphorous plating bath also may be subjected to a periodic or continuous filtration treatment to reduce the level of contaminants therein. Replenishment of the constituents of the bath may also be performed, in some embodiments, on a periodic or continuous basis to maintain the concentration of constituents, and in particular, the concentration of nickel ions, copper ions, and hypophosphite ions, as well as the pH level within the desired limits.

The electroless nickel-copper-phosphorous alloy coated substrate so formed can be removed from the electroless nickel-copper-phosphorous plating bath and rinsed, for example, with deionized water.

In some embodiments, the electroless nickel-copper-phosphorous alloy coating so formed includes at least about 30% by weight Cu; about 5% to about 15% by weight P; and the balance Ni, with incidental impurities.

In some embodiments, the coating can include at least about 40%, at least about 50%, or at least about 60% by weight Cu and have a thickness of at least about 50 microinches. The coating can be formed using the bath and/or the method described herein.

In some embodiments, the coating can be biocidal, antimicrobial, and/or destroy or inhibit the growth microorganisms, such as bacteria, fungi, and viruses.

In other embodiments, the coating can be antipathogenic and destroy or inhibit the growth of bacteria, such as Staphylococcus aureus or methicillin-resistant Staphylococcus aureus.

In still other embodiments, the coating can inhibit bio-film formation.

The following examples are illustrative of the electroless nickel-copper-phosphorous plating solutions of the invention. Unless otherwise indicated in the following example, in the written description and in the claims, all parts and percentages are by weight, temperatures are in degrees Fahrenheit and pressure is at or near atmospheric pressure.

### Example

An autocatalytic electroless plating system was developed that can provide a uniform coating on a substrate that consists of a nickel-copper-phosphorus alloy that exhibits enhanced antipathogenic properties. In other embodiments, the electroless plating system provides a uniform coating on a substrate that consists essentially of a nickel-copper-phosphorus alloy. What is meant by “consisting essentially of” is that the alloy is free of any components that would have adverse effect on wearability of the alloy deposit and/or adverse effect on the antipathogenic properties of the alloy deposit. In other embodiments, the alloy comprises or includes a nickel-copper-phosphorous alloy. The autocatalytic electroless plating system enables complete encapsulation of the substrate, high wearability so that it can withstand much contact, and copper for enhanced antipathogenic properties.

The following example shows the improved antipathogenic properties of the alloy.

An electroless nickel-copper-phosphorus bath was prepared comprising:
3 g/L Nickel (as provided by nickel sulfate hexahydrate)
15-35 g/L hypophosphorous reducing agent (as provided by sodium hypophosphite monohydrate)
400 to 1,000 ppm Copper (as provided by copper sulfate pentahydrate)

The temperature of the bath was maintained at 160-195 F and the pH of the bath was adjusted to and maintained within a range of 8-9. The plating time of the substrate was one hour and the plating rate was about 0.4 mils/hour.

The Ni concentration was maintained constant and the copper concentration was varied to achieve to produce both a 40 and 60% copper concentration in the resultant alloy. 60% copper is the minimal recognized concentration where optimal antipathogenic properties occur. However, the inventors of the present invention surprisingly discovered that there is considerable biocidal activity even at lower copper percentages.

Two controls were used during the testing. The first was a control done only on plastic that should have had no impact on the bacteria. The second was a standard electroless nickel-phosphorous deposit. Two separate standard testing bacteria were used:
Staphylococcus aureus ATCC 6538P
Methicillin-resistant Staphylococcus aureus (MRSA) ATTC 33592

### Test Procedure Summary

The test organism was adjusted and diluted to obtain the starting inoculum concentration of 2.5-10×10⁵ CFU/mL. The control was tested in triplicate at Time=0 and Time=2 hours. The test samples were tested in triplicate at Time=2 hours. Each sample piece was placed in a sterile Petri dish, inoculated and then covered with the sterile plastic in order to spread the inoculum evenly over the sample surface and hold its place. The sample were incubated at 35° C. and a relative humidity of at least 90%. At the appropriate time the neutralizing broth was added to each sample, placed onto a shaker and mixed thoroughly to facilitate the release of the inoculum from the sample surface. Serial dilutions of the neutralizing broth containing the inoculum were plated. All plates were incubated at 35° C. for 24-48 hours. After incubation, bacterial colonies were counted and recorded. The results are found in the “Test Results” section below. These results pertain only to the samples tested.

### Test Variables

| | |
|---|---|
| Test Organism: | Staphylococcus aureus ATCC 6538P |
| | Methicillin-resistant S. aureus |
| | (MRSA) ATCC 33592 |
| Sample Size: | 50 mm × 50 mm |
| Method of Sterilization/ | None |
| Pre-Cleaning: | |
| Control Sample: | Enova 949 - Control sample submitted |
| | by customer |
| Film Used: | 40 mm × 40 mm s 0.05 mm plastic pieces |
| | cut from sterile Whirlpak ™ bags |
| Dilution Medium Used: | Sterile dilute nutrient broth per standard |
| Neutralizing Broth Used: | D/E Neutralizing Broth |
| Amount of Neutralizing | 10 mL |
| Broth: | |
| Starting Inoculum | S. aureus ATCC#6538P: 3.1 c 10⁵ |
| Concentration: | S. aureus (MRSA) ATCC 33592: |
| | 3.2 × 10⁵ |
| Amount of Inoculum | 0.4 mL |
| Contact Time: | 2 hours |
| Deviations from Standard | Additional test organism Contact time |
| Test Method: | changed from 24 hours to 2 hours |

### Test Results

| | | | # of | % of | % |
|---|---|---|---|---|---|
| | Anti- | | nonviable | Nonviable | improvement |
| | microbial | Bacteria | Bacteria | bacteria | over Control |
| | Activity | Survival | Colonies | cells | (949) |
| MRSA (ATCC#33592 | | | | | |
| # of Viable Bacteria | 4.17 | 14791.08388 | | | |
| (Round 2) | | | | | |
| STD EN Control | 1.82 | 66.0693448 | 14725.01454 | 99.55% | — |
| Ni—Cu—P (40% Cu) | 0.55 | 3.548133892 | 14787.53575 | 99.98% | 95% |
| Ni—Cu—P (60% Cu) | 0.47 | 2.951209227 | 14788.13267 | 99.98% | 96% |
| Untreated Plastic | | | # of | | |
| Control | | | surviving | | |
| (Lab Provided) | | | colonies | | |
| # of Viable colonies at | 4.23 | | 16982.43652 | | |
| 0 hr | | | | | |
| # of Viable colonies at | 4.27 | | 18620.87137 | | |
| 2 hr | | | | | |
| S. Aureus | | | | | |
| (ATCC#6538P) | | | | | |
| # of Viable Bacteria | 4.15 | 102.33 | | | |
| (Round 2) | | | | | |
| STD EN Control | 2.01 | 69.18 | 14023.05 | 99.28% | — |
| Ni—Cu—P (40% Cu) | 1.84 | 5.37 | 14056.19 | 99.51% | 32% |
| Ni—Cu—P (60% Cu) | 0.73 | | 14120.01 | 99.96% | 95% |
| Untreated Plastic | | | # of | | |
| Control | | | surviving | | |
| (Lab Provided) | | | colonies | | |
| # of Viable colonies at | 4.18 | | 15135.61248 | | |
| 0 hr | | | | | |
| # of Viable colonies at | 4.31 | | 20417.37945 | | |
| 2 hr | | | | | |

The results show that there was indeed an improvement in antipathogenic response of the coatings that contain copper as compared to the just nickel-phosphorous control. In the case of the Staphylococcus aureus ATCC 6538P there was a 32% improvement to the efficacy with 40% copper in the alloy and a 95% improvement at 60%. In fact, the 60% alloy had nearly a 100% disinfection rate at just 2 hour exposure.

The alloys were even more effective against the Methicillin-resistant Staphylococcus aureus (MRSA) ATTC 33592. The 40% copper alloy had a 95% improvement over control and the 60% alloy achieved 96% improvement. Both provided 99.98% elimination of pathogens after 2 hours exposure. The surface essentially self-disinfected.

In both cases, the control in just plastic contained more bacteria after the 2-hour incubation. This is verification that the test was valid (that the bacteria did not just spontaneously expire).

From the above description of the invention, those skilled in the art will perceive improvements, changes and modifications. Such improvements, changes, and modifications are within the skill of the art and are intended to be covered by the appended claims. All patent publications and references cited in the present application are herein incorporated by reference in their entirety.

What is claimed is:

1. An aqueous electroless nickel-copper-phosphorous plating bath for forming an antipathogenic coating on a substrate, the plating bath comprising:
about 2.0 g/l to about 8.0 g/l Ni;
about 150 ppm to about 1500 ppm Cu;
about 10 g/l to about 50 g/l hypophosphorous reducing agent;
wherein the electroless nickel-copper-phosphorous bath provides a uniform electroless nickel-copper-phosphorous deposit on the substrate that is antipathogenic and/or oligodynamic.

2. The bath of claim 1, wherein the concentration of Ni in the bath relative to the concentration of Cu is such that the electroless nickel-copper-phosphorous deposit includes about 30 wt. % to about 85 wt. % Cu.

3. The bath of claim 1, wherein the Ni concentration in the plating bath is about 3 g/L to about 6 g/L.

4. The bath of claim 1, wherein the Ni is provided in the bath by dissolving a water soluble nickel salt in the bath.

5. The bath of claim 4, wherein the water soluble nickel salt is selected from nickel sulfate, nickel chloride, nickel methane sulfonate, nickel sulfamate, nickel fluoroborate, or combinations thereof.

6. The bath of claim 1, wherein the Cu concentration is about 400 ppm to about 1000 ppm.

7. The bath of claim 1, wherein the Cu is provided in the bath by dissolving a water soluble copper salt in an aqueous solution.

8. The bath of claim 7, wherein the copper salt is selected from copper sulfate, copper chloride, copper methane sulfonate, copper sulfamate, copper fluoroborate, or combinations thereof.

9. The bath of claim 1, having a neutral to alkaline pH.

10. The bath of claim 9, having a pH of about 8 to about 9.

11. The bath of claim 1, comprising about 10 g/l to about 50 g/l of at least one organic acid complexing agent.

12. The bath of claim 11, wherein the organic acid complexing agent comprises a carboxylic acid selected from the group consisting of malic acid, lactic acid, succinic acid, and combinations of the foregoing.

13. The bath of claim 1, further comprising at least one of a chelating agent, stabilizer, or pH buffer.

14. The bath of claim 1 consisting essentially of water, about 3 g/L to about 6 g/L of Ni, about 10 g/L to about 50 g/L hypophosphorous reducing agent, about 400 ppm to about 1000 ppm Cu, less than about 25 g/L of a combination of organic acid complexing, agents selected from the group consisting of malic acid, lactic acid, and succinic acid, an optional stabilizer, and an optional pH adjuster, wherein the bath has a pH of about 8 to about 9.

15. A method of forming an electroless antipathogenic coating on a surface of a substrate, the method comprising:
providing a substrate,
contacting a surface of the substrate with the aqueous electroless nickel-copper-phosphorous plating bath of claim 1.

16. The method of claim 15, wherein surface of the substrate is contacted with the aqueous electroless nickel-copper-phosphorous plating bath by immersing at least a portion of the substrate in the bath.

17. The method of claim 15, wherein the bath has a temperature during electroless plating of the surface of the substrate of about 160° F. to about 195° F.

18. The method of claim 15, wherein the surface is in contact with the bath for at least 1 hour.

19. The method of claim 15, wherein the antipathogenic coating has a thickness of at least about 50 microinches.

20. An electroless nickel-copper-phosphorous coating, comprising:
at least about 30% by weight Cu;
about 5% to about 15% by weight P;
and the balance Ni, with incidental impurities.

21. The coating of claim 20, comprising at least about 40 by weight Cu.

22. The coating of claim 20, having a thickness of a thickness of at least about 50 microinches.

23. The coating of claim 20, wherein the coating is antipathogenic, antimicrobial, and/or inhibits bio-film formation.

24. The coating of claim 23, wherein the coating is antipathogenic to bacteria comprising at least one of Staphylococcus aureus or methicillin-resistant Staphylococcus aureus.

25. (canceled)
